(19) United States
(12) Patent Application Publication
(10) Pub. No.: US 2025/0241973 A1
(43) Pub. Date: Jul. 31, 2025

(54) EXTRACTIVE PROCESS FOR OBTAINING AN OIL BASED ON PHYTOCANNABINOIDS

(71) Applicants: Daniele TARTAGLIA, Lanciano (CH) (IT); Edoardo Maria TONELLI, Lanciano (CH) (IT); Lavinia Maria TONELLI, Lanciano (CH) (IT)

(72) Inventors: Daniele TARTAGLIA, Lanciano (CH) (IT); Alessandro TONELLI, Lanciano (CH) (IT)

(21) Appl. No.: 18/856,483

(22) PCT Filed: Apr. 3, 2023
(86) PCT No.: PCT/IB2023/053354

§ 371 Date: Oct. 11, 2024

(30) Foreign Application Priority Data
Apr. 14, 2022 (IT) 102022000007457

(51) Int. Cl. A61K 36/185

(52) U.S. Cl. CPC A61K 36/3482; A61K 2236/15; A61K 2236/333; A61K 2236/35; A61K 2236/37; A61K 2236/53

(57) ABSTRACT

Extractive process for obtaining an oil based on phytocannabinoids, said oil being selected from olive oil, extra virgin olive oil, seed oil in general, hemp seed oil and combinations thereof, said process using as biomass starting with dried flowers and leaves of the Cannabis plant, said process providing for the implementation in sequence of the following steps: kneading, extraction, decanting/separation, filtration; said process being characterized in that it jointly uses as extraction solvent water as polar solvent and an oil selected from said olive oil, extra virgin olive oil and hemp seed oil, seed oil in general and combinations thereof such as apolar solvent, said starting biomass including substances such as CBD, CBDA, CBG, CBGA, THC, THCA, THCV, THCVA, terpenes, fat-soluble cannabinoids.

### FIELD OF THE INVENTION

The present description relates to the medical and nutraceutical fields. More in detail, the present invention relates to a particular extractive process for the preparation of an edible oil, typically olive oil, comprising cannabinoids and terpenes, and in particular CBD and CBG. Said process, in addition to allowing the preparation of a product which is used in the treatment of various pathologies, proves to be particularly advantageous in terms of cost-effectiveness for its realization as well as for the high yields of the product obtained directly from said process.

### PRIOR ART

Cannabis or hemp is a genus of flowering plants in the Cannabaceae family. According to some, it includes a single species, Cannabis sativa, historically the most widespread plant in the West, which in turn includes various varieties and subspecies; according to others, however, three species are distinguished, C. sativa, C. indica and C. ruderalis. Hemp is an annual herbaceous plant whose height ranges from 1.5 to 6 meters, although some subspecies have final heights which ranges from 0.5 (ruderalis) to 5 (sativa) meters. It has a long taproot and a stem, erect or branched, with resinous, angled, sometimes hollow outgrowths, especially above the first pair of leaves. The leaves are petiolate and provided with stipules; each of them is palmate, composed of 5 to 13 lanceolate leaflets, with toothed-serrated margin, with sharp points up to 10 cm long and 1.5 cm broad; in the lower part of the stem, the leaves are opposite, while in the upper part they tend to grow alternately, especially after the ninth/tenth node of the plant, i.e. after sexual maturation (after the initial vegetative phase, popularly known as “rising”). Hemp plants are both monoecious (useful for the production of seeds for food use) and dioecious. The male flowers (staminiferous) are grouped in terminal panicles and each has 5 stamens. The female flowers (pistilliferous) are grouped in groups of 2-6 at the axils of bracts forming short ears; each shows a membranous calyx tightly enveloping a superior and unilocular ovary, surmounted by two styles and two stigmas.

Marijuana is the variant of hemp rich in THC, a psychoactive substance obtained from the dried inflorescences of female hemp plants (Cannabis). All varieties of hemp contain various psychoactive substances, both narcotic and non-narcotic, in variable concentrations and proportions, the main one being delta-9-tetrahydrocannabinol (commonly called THC), which make the plant illegal in many countries. However, there are legally cultivable varieties, for which the limit to this content is set by law. The medical use of Cannabis it has a millennial history shared by many cultures in the world. More than 750 different substances have been identified in the plant, 113 of which have a structure similar to the main cannabinoids, some of which have opposing actions, the concentration and distribution of which can vary considerably according to the genetic profile of the plant and the stress to which it is subjected during its growth. In modern medicine, rather than the combustion and inhalation of the dried inflorescences of the plant, a typical method for the recreational consumption of the plant, preparations based on synthetic cannabinoids or titrated extracts from the plant are studied and used, with which it is possible to carry out adequate clinical trials. The uses of Cannabis for medical use are present in controlled clinical trials and observational studies, in systematic reviews and in meta-analyses of the indexed international literature. The therapeutic efficacy of Cannabis is not well defined, some research draws conflicting conclusions and often the evidence is scarce or mediocre. In consideration of the scientific evidence produced, it can be stated that the medical use of Cannabis cannot be considered a therapy in the strict sense, but a symptomatic treatment to support standard treatments, when the latter have not produced the desired effects, or have caused intolerable secondary effects, or require dosage increases which could lead to the appearance of side effects. The laws of the different States that allow the prescription and use of Cannabis for medical use can authorize different therapeutic indications. Typically these concern: analgesia in pathologies involving spasticity associated with pain (multiple sclerosis, spinal cord injury) resistant to conventional therapies; analgesia in chronic pain (with particular reference to neurogenic pain), in which treatment with non-steroidal anti-inflammatory drugs or with cortisone or opioid drugs has proved to be ineffective; the antikinetic and antiemetic effect in nausea and vomiting, caused by chemotherapy, radiotherapy, HIV therapies (which cannot be obtained with traditional treatments); the appetite-stimulating effect in cachexia, anorexia, loss of appetite in cancer or AIDS patients and in anorexia nervosa (which cannot be achieved with standard treatments); the hypotensive effect in glaucoma resistant to conventional therapies; the reduction of involuntary body and facial movements in Tourette syndrome that cannot be achieved with standard treatments; the bronchodilator effect in asthma attacks.

In particular, there is conclusive or substantial evidence that Cannabis or cannabinoids are effective: for the treatment of chronic pain in adults (Cannabis); as an antiemetic in the treatment of nausea and chemotherapy-induced vomiting (oral cannabinoids); to improve symptoms of spasticity of multiple sclerosis (oral cannabinoids).

Further therapeutic indications are aimed at: short-term improvement of the quality of sleep in subjects with sleep disorders associated with obstructive sleep apnea syndrome, fibromyalgia, chronic pain and multiple sclerosis (cannabinoids, mainly nabiximols, Cannabis and oral cannabinoids).

In summary, it can be summarized that Hemp or Cannabis (the names by which it is known vary from country to country and are sometimes referred to the presence of THC, such as for example the term “Marijuana”), is a plant, belonging to the family of Cannabaceae used for centuries by the populations of the world for its various uses: from those sometimes controversial, linked to its psychotropic characteristics, to therapeutic ones, such as those concerning the field of application to which the present invention relates. In fact, different products can be obtained from the same plant, depending on whether the variety contains a greater or lesser quantity of THC (the psychotropic component, not legal for recreational purposes, but regularized for therapeutic purposes), or on the contrary is rich in phytocannabinoids such as those used in the products according to the present invention, i.e. CBD, for which there is recent interest in the preparation of CBD oils, crystals, CBD capsules, oils for animals, or for CBG-based products (cannabigerol), a non-psychotropic phytocannabinoid, perfectly legal, with excellent characteristics.

Equally recent is in fact the increasingly felt interest by the population towards CBD oil and, specifically, for the benefits it brings, it is no coincidence that different scientific studies are carrying out research on the effects and possible applications. The interest of the scientific community for the therapeutic potential of CBD oil is therefore growing every day. The reasons are primarily due to the fact that this oil has already been used in various scientific studies, for the treatment of numerous health problems and is now recognized as one of the main elements of the so-called “Therapeutic Cannabis”.

The techniques that are mainly used today are typically extraction techniques, among which the most used ones are listed below. In particular, the different methods are able to divide the plant material of the Cannabis plant into different parts, or extracts, which contain different chemicals. In the case of Cannabis (Sativa L. with a high concentration of non-psychoactive cannabinoids or Indica with a much higher concentration of THC, a psychoactive molecule), extraction techniques are often used to isolate specific desired components; in fact, the plant contains more than one hundred cannabinoids, including cannabidiol (CBD) and tetrahydrocannabinol (THC), CBG (cannabigerol), THCV and so on. But a producer may also be interested in creating a single extract that comprises many desirable components of Cannabis; this is called whole plant extract.

In addition to the well-known cannabinoids, scientists have identified more than 500 different chemicals in Cannabis, including terpenes, flavonoids, etc. Extractive techniques are also used to isolate individual substances of interest such as CBD, THC, CBG or terpenes and flavonoids. In more detail, the extracts are obtained through the use of solvents. In the extraction industry, the most popular solvent types are butane (BHO), carbon dioxide (CO₂), ethanol (ethyl alcohol), and propane; all valid options for Cannabis extractions.

As far as butane extraction is concerned, it must be said that this process leads to the formation of a Cannabis concentrate, commonly known as BHO, which is produced using butane as a solvent. This type of gas is used because it does not remove the parts soluble in water (for example, chlorophyll). Another type of solvent, such as ethyl alcohol, would quickly release the chlorophyll, making the oil more bitter and dark green in color. The extractive process aims to separate resin and trichomes (in which terpenes and cannabinoids are found) from the rest of the plant content of the Cannabis. There are several variables (especially the temperature) that determine the final consistency of the BHO, depending on this consistency the obtained extraction will receive different names. To make BHO, one needs Cannabis, liquid butane, and a pressurized, heated hose. The butane is then removed using vacuum evaporation. The vacuum condition transforms the liquid butane into vapor, making it easier to remove.

Using this extraction method, a very tasty product is obtained with a significant terpene profile whose concentration of cannabinoids can reach 75-90%. This makes BHO a very popular choice among medical Cannabis users suffering from chronic pain, sleep disorders, and other ailments.

Butane is easily flammable when it is in the gaseous state, caution is therefore recommended in the temperature used, given the risk of gas explosion. Furthermore, a system of this type should include circulators that remove and recycle the butane, in order to have a certain environmental compatibility of the process. This removal process should eliminate any butane residue in the final extract. In any case, analytical tests should be performed to ensure the removal of butane as it is highly toxic to humans.

Instead of butane hash oil, some manufacturers opt to produce propane hash oil. This method uses liquid propane rather than butane. In doing so, the high pressure keeps the propane in the liquid state and the extraction takes place at lower temperatures, due to the fact that its boiling point is lower than that of butane.

The extraction temperature determines the components that are extracted from Cannabis. So, these two extraction methods—butane and propane—produce different concentrates. In some cases, these two processes may be combined to create a product with a broader chemical profile.

In any case it should be emphasized that the main critical issues associated with the extraction of cannabinoids using propane or butane is extremely dangerous and not very respectful of the environment, and it is not easy to remove all the residues of these substances from the final extract.

This process concentrates all cannabinoids, including THC beyond the limits of 0.5% up to 2%- 4%, even if starting from a Cannabis biomass that initially has 0.2% THC; for this reason, it is necessary to have the appropriate authorizations required by the laws of each country in which it is performed.

As far as extraction using CO₂ is concerned, first of all it must be said that CO₂ is a pure chemical substance which is found in nature and which leaves no traces of residues. This is why supercritical extraction is a standard method that is already being used in the food, dry cleaning and herbal supplement industries. It is also a common food additive. CO₂ extractors use a combination of pressure and temperature to force carbon dioxide past its “critical point”. This takes place at a pressure of 100 atmospheres and temperatures of about 27° C.

Once forced into its supercritical state, CO₂ becomes an organic and environmentally friendly solvent. It boasts non-flammable, non-toxic and inert properties, yet effectively extracting compounds such as cannabinoids, terpenes and other Cannabis compounds. The CO₂ extraction process allows the components to be extracted with almost no toxicity; a pressure vessel containing Cannabis is used, then the supercritical CO₂ is inserted which is pumped through a filter in which, once the pressure is lowered, it is separated from the plant material. Subsequently, the supercritical CO₂ evaporates and dissolves into cannabinoids. This extraction method is very effective, in fact everything is removed from the plant material, including waxes, pigments and cell wall fragments. So it is necessary to carry out the so-called winterization or dewaxing process to obtain safe and consumable extracts. This process consists in mixing the substance obtained with the supercritical CO₂ extraction apparatus with ethanol, cooling the whole, filtering to eliminate the mass of unusable waxes and finally evaporating the ethanol.

The equipment cost for this method is significantly higher than extraction with alcohol as a solvent, but it produces higher yields and less valuable material is lost. Furthermore, this method can be used to extract specific components by changing temperature, pressure or running time—or rather a combination of these three variables.

This process concentrates all cannabinoids, including THC beyond the limits of 0.5% up to 2%- 4%, even if starting from a Cannabis biomass that initially has 0.2% THC; for this reason, it is necessary to have the appropriate authorizations required by the laws of each country in which it is performed.

Furthermore, it is extremely expensive both in terms of plant costs and in terms of management and maintenance costs, and the result of the extraction process is not a CBD oil but an extract that must be further processed with other synthesized solvents. CO₂ extraction is done using large and extremely expensive industrial units, made up of a series of chambers and tubes. Due to their size and cost, only companies willing to make a large investment can use this technology to create top-notch Cannabis extracts.

CO₂ extractors remain bulky and consist of many parts, including CO₂ tanks, pumps, extraction vessels, separators, and a collection vessel.

Only fat-soluble molecules are transported in the extract, not water-soluble ones such as flavonoids.

This form of extraction is however efficient, ecological (it does not pollute as the solvent is continuously circulated in the plant), safe for health, non-toxic, non-flammable. The decarboxylation of cannabinoids is carried out with the classic known processes, bringing the Cannabis flowers to temperatures above 110-120° C., before the extractive process. A further known extractive method is the extraction in alcohol.

Also known as Cannabis oil, hemp oil, Phoenix tears and Rick Simpson Oil (RSO), whole plant Cannabis oil can be administered orally or applied directly to the skin. The sublingual route of application is the preferred treatment method for many. Intake by this method, i.e. via the mucous membrane, is a convenient way of intake that provides rapid and effective absorption directly into the systemic circulation, thanks to the increased bio-availability of cannabinoids.

True whole plant oil is obtained from the flowers of the female Cannabis plant and includes many cannabinoids, including THC, CBD, CBN and others—as well as terpenes and other components. Today, many companies sell their own version of Rick Simpson oil: some of them high in THC, while others contain only non-psychoactive components such as CBD. Alcohol extraction of Cannabis has a history of hundreds of years. Cannabinoids have excellent solubility in types of alcohol such as ethanol and isopropanol, provided the water content in the alcohol is low. Cannabis tinctures made with ethanol are part of the pharmacopoeia and, before prohibition, were readily available for many ailments. Using ethanol to extract the beneficial components of the Cannabis plant is a method that is considered much safer and much simpler than others. Obtaining an extract of this type takes a few minutes in bath in the chosen solvent. Extracting in alcohol requires the use of anti-detonation systems due to its flammability and is in any case an expensive solvent. Furthermore, both chlorophyll, resins, flavonoids, CBD, CBG, THC, terpenes are concentrated in a single extract without any selection. This process concentrates all cannabinoids, including THC beyond the limits of 0.5% up to 2%- 4%, even if starting from a Cannabis biomass that initially has 0.2% THC; for this reason, it is necessary to have the appropriate authorizations required by the laws of each country in which it is performed. Recently, the so-called “Rosin” has gained a lot of popularity in the medical cannabis community. It is a solid form of resin that is obtained by applying pressure and heat, usually with a pneumatic heat press (or even with a hair straightener when the batch is small), to vaporize the volatile liquid terpene components. Rosin is a concentrate. It is made using heat and pressure instead of the solvents mentioned above. Since no solvents or expensive machinery are needed, rosin is much cheaper to produce. The process required to create this concentrate is also much less complicated than solvent extraction and can even be done at home.

Difficult to apply industrially, especially for high productions, this method concentrates all cannabinoids, including THC beyond the limits of 0.5%, even if starting from a cannabis biomass that initially has 0.2% THC; for this reason, it is necessary to have the appropriate authorizations required by the laws of each country in which it is performed.

In all the processes listed and known for the extraction of cannabinoids, sintering takes place at very cold temperatures, below 0° C., in the process described below, however, it may also take place at room temperature.

The object of the present invention, described in detail hereinafter, is to propose a particular method for the extraction of active ingredients from Cannabis, and in particular of phytocannabinoids, for the preparation of an edible oil for therapeutic use. The method according to the present invention is particularly advantageous both from a procedural and an economic point of view and allows a multiplicity of by-products to be obtained which may also be used for multiple applications. Said and further advantages will become more apparent in the following description of the invention.

### DESCRIPTION OF THE INVENTION

The present description relates to a particular Cannabis extractive process for obtaining a plurality of extraction products of interest for various applications and, in particular, for obtaining a phytocannabinoid-based oil for therapeutic and food use.

More in detail, the present invention relates to a solid-liquid extraction method which is first of all characterized in that it uses both a polar solvent and an apolar solvent as extraction solvent.

Even more in detail, the extraction process according to the present invention provides the joint use of the polar solvent water, and an apolar solvent immiscible with water, to obtain: the controlled decarboxylation in water of the acid molecules present in the solid matrix of Cannabis biomass, and three solutions/suspensions, one containing the molecules similar to the polar solvent and the two other ones similar to the apolar solvent and present in the solid matrix of Cannabis.

The starting solid matrix used in the method in question is represented by biomass of whole or shredded flowers and leaves with particle size between 1 mm and 10 mm. The biomass may also be introduced as intact flowers and leaves since during kneading, such biomass is broken up and reduced into smaller parts. It is of interest to point out that it is important to avoid the presence of large branches. The solid matrix must not have a powdery character, as a powdery solid matrix would make the process less efficient in terms of quantity, quality and in terms of quantity of cannabinoid molecules transported from the solid matrix to the apolar solvent represented by olive oil, extra virgin olive and/or hemp seed oil.

The type of biomass of Cannabis, or hemp to be used depends on the concentration of the cannabinoid that one wishes to have inside the apolar solvent chosen at the end of the process, in particular at the end of the extraction process four products are obtained which for simplicity, during the present description will be indicated as sub-products A, B, C and product D. The latter represents the one of greatest interest according to the present invention.

It is interesting to note that if a CBD oil is desired, a biomass with a high content of CBD+CBDA should be chosen, if a CBG oil is desired, a solid matrix with a high content of CBG+CBGA should be used, or THC+THCA, if an apolar solvent with a high concentration of THC, or other fat-soluble cannabinoid molecules, is desired. By choosing the appropriate solid starting matrix, it is therefore possible to obtain a concentrated oil with CBD, or CBG, or THC, or any other desired fat-soluble cannabinoid.

As mentioned above, the polar solvent of choice is water, given its cheapness and its chemical-physical behavior in contact with oils and waxes, and in particular with the solid Cannabis matrix: water gives a unique elasticity and plasticity to the Cannabis biomass. Furthermore, the water is decidedly effective during the decantation process given its clear separation from the apolar solvent used, olive oil and/or hemp seed oil. Water also allows most of the waxes and vegetable fats found in Cannabis resins to winterize at room temperature. The water also allows the separation of sugars-proteins-enzymes-flavonoids and other water-soluble substances which will remain more dissolved in the water. The apolar solvent of choice is extra virgin olive oil and/or hemp seed oil. However, other edible oils may be used such as sunflower, peanut or olive oil, flax, corn, soy, rapeseed, sesame, palm or other seeds. The steps of the method according to the present invention provide in sequence: Kneading; Extraction; Decanting; Filtration.

All the elements of the machines in contact with the solid matrix and/or with the solvents are in food grade stainless steel and the finishing standard of the machines is in pharmacological GMP.

Kneading is the first step of the process—horizontal or vertical—, in which the solid matrix is mixed with water and olive oil and/or hemp seed oil and/or other non-polar solvent immiscible in water.

The kneading step leads to the rehydration of the dry matrix, and decarboxylation or not of molecules such as THCA, CBDA, CBGA, or all those cannabinoids present in their acid form within the Cannabis biomass.

As is known, the kneading machine is a double-walled jacketed container in which a cooling fluid flows, typically water, the temperature of which can be regulated from the outside, from a minimum of 0° C. up to a maximum of 95° C. Inside the kneading machine there are stirrers capable of uniformly mixing the materials introduced therein, so that the temperature of the internal walls is uniformly transmitted to the whole compound in the mix. The mixing speed, the operating temperature and the mixing time are externally adjustable. After introducing the solid matrix, polar solvent and then apolar solvent, it is possible to set a certain desired kneading cycle.

The kneading cycle must be established according to the type of cannabinoid molecule which mainly characterizes the starting solid matrix (cannabinoids have different melting temperatures, generally between 40° C. and 70° C.) and which one wishes to bring into the apolar solvent. Furthermore, this kneading cycle depends on whether or not the cannabinoids in their acidic or decarboxylated form are desired in the apolar solvent. If non-decarboxylated cannabinoid molecules are desired, the kneading will take place at temperatures which allow the dissolution of the cannabinoids but not their decarboxylation, generally temperatures between 40°° C. and 70° C. and for a reduced kneading time. Otherwise it will be necessary to go beyond 70° C. during kneading and lengthen the kneading times even up to 4 hours (or more), to have completely decarboxylated cannabinoids in the apolar solvent at the end of the process.

The first material to be introduced into the kneader is the chosen solid matrix of Cannabis. Subsequently, water is introduced, which can be hot or cold, even at 0° C., or in the form of ice. Ice freezes the fat-soluble molecules present in the dry matrix and favors their detachment from the solid matrix (fiber and plant cells). Ice also favors the crushing of coarser solid matrices. Cold water, close to 0 (zero), may also be used, not necessarily in the form of ice, if the particle size of the matrix is between 0.5 mm and 7 mm, and if it is not of interest to decarboxylate the molecules. In other words, if desired at the end of the process, in the non-polar solvent molecules that are not decarboxylated, but present in their acid form, then cold water will be used in the kneading process, or in any case the kneading temperature can never exceed those limits of temperature and time of kneading, which lead to the decarboxylation of cannabinoid acid molecules, such as CBDA, CBGA, THCA and so on. So if one intends to obtain non-decarboxylated cannabinoids at the end of the process, the water temperature cannot exceed 70° C., and the kneading time will be reduced to about 1.5 hours. If instead it is of interest to decarboxylate the acid molecules present in the matrix it will be necessary to introduce water from 0° C. to 70° C., and then let it knead for a time between 1 and 4 hours or more, with temperatures between 70 and 95 degrees, however not beyond the boiling point of water. In this process, a slow decarboxylation takes place, which takes hours, unlike the currently known decarboxylation processes which take place at operating temperatures higher than 100-120° C., and which take place in decidedly shorter times than those characterizing the method according to present invention. The water introduced is absorbed by the particular spongy structure of Cannabis leaves and flowers, and as kneading takes place, it will be in close and uniform contact with the liposoluble cannabinoid molecules present in the solid matrix. As the kneading temperature increases, the water transfers this temperature to the cannabinoid molecules, so that when the temperature reaches around 75° C., the decarboxylation of the cannabinoids CBDA, or CBGA or THCA begins. When the kneading temperature reaches 95-100° C., the decarboxylation process increases and remains constant for about 3/4 hours. This is a slow decarboxylation, as normally the same, in current known processes, takes place above 100/120° C., in very short times, 40/60minutes. Although the decarboxylation times in the process according to the present invention are relatively long, the advantages lie in that by working at relatively low temperatures, the onset of degradative processes is reduced. Therefore, the control of temperatures and kneading times chosen favor the integrity of the entire phytocomplex of molecules present in Cannabis flowers, to the advantage of the final product, oil with CBD, CBG, or THC, to be obtained at the end of the process.

After 3/4 hours of kneading with water, it is therefore possible to reduce the operating temperature between 60° C. and 85° C., which depends a lot on the melting temperature of the cannabinoid most present in the solid matrix and to be bound to the molecules of oil, and introduce the apolar solvent, for example olive oil and then continue kneading for another 40/60 minutes.

Before introducing the olive oil (or chosen apolar solvent), during the kneading step, it is possible to drain most of the water with which the Cannabis biomass is kneading, and introduce new water, even several times; the temperature of the new water introduced will be between 40° C. and 95° C. The purpose of this replacement of water during kneading has the objective of removing a large part of sugars and proteins from the initial mass and of many water-soluble substances from the entire compound, and immediately separating from the compound sugars, vegetable proteins, flavonoids, dissolved enzymes and other water-soluble substances.

For example, it is preferable to start kneading with water, reaching an internal temperature of 50° C., and removing, for example, 3 liters of first kneading water, and reintroducing another 3 liters of new water at a temperature of 50-60° C., in order to maintain a constant internal temperature of the compound being kneaded.

This water extracted from the kneader is full of sugars, vegetable proteins, flavonoids and water-soluble substances, and will be added at the end of the process to what according to the present invention is indicated as Process by-product B.

After substantially “washing” the compound in the kneader, even several times, the apolar solvent is introduced to then continue with the process steps.

At this point it is possible to transfer the compound into the extractor. Briefly, the previous kneading step may require that the solid matrix be inserted at room temperature, that the water is inserted at ambient temperature, typically at 25° C. or at about 25° C. (or in the range 0° C.-70° C.), that it is allowed to be kneaded until the temperature of the solid matrix compound+water reaches at least 90-95° C. (in case one wishes to decarboxylate the molecules), and that it is continued to be kneaded for about 3-4 hours, until almost complete decarboxylation of the CBDA, that during this kneading step the water may also be changed several times.

Subsequently, the apolar solvent into which the fat-soluble molecules are to be transferred is introduced into the kneader, in this case extra virgin olive oil, and the mixture continues to be kneaded for an interval of time ranging from 30 minutes to 70 minutes, preferably for 60 minutes and even more preferably for 50 minutes, at a temperature between 66 and 85° C., preferably 75-79° C. Finally, the kneading compound can be transferred to the extractor.

As regards the following extraction step, it is of interest to note that, in the process according to the present invention, the extractor used is, preferably but not necessarily, an apparatus comprising a hydraulic press which pushes a piston inside a perforated cylinder; the holes are uniformly distributed on all the walls of the cylinder and preferably have dimensions ranging from 0.5 to 3 mm, more preferably 1.6 mm. The cylinder is inserted on a cylindrical base, which is also perforated like the cylinder. This perforated base rests on a further base with side walls (collection base) and a single outlet hole for collecting the solutions. Around the perforated cylinder there is a thermal jacket of removable walls fixed on the collection base. The thermal jacket can be double-walled with water heating in order to manage the temperature of the extraction micro-environment, which is created when this jacket is closed. The diameter of the extraction cylinder varies according to the productivity to be predefined. Typically on a laboratory scale it varies from 20 to 40 cm, and its height from 20 to 80 cm. The non-miscibility of the two solvents, and the variations in viscosity and density from the beginning of the process with the gradual increase in temperature during kneading, are fundamental for having an extraction process with high yields. By first introducing the water into the kneading machine, the same is absorbed in a completely natural way by the cannabis biomass (considering its vegetable microstructure), which recovers an enormous elasticity, compared to its dry state. The plant structure of the leaves and of the Cannabis flower, whose lipophilic molecules (CBD, CBG, THC, waxes, terpenes) are found in the external part of the floral and foliar surface, at the end of flower maturation, the high pressure exerted by the extractor, the use of a polar solvent such as water easily absorbed by the vegetable part of flowers and leaves, the use of another non-polar solvent such as olive oil or hemp seed oil, make this process possible, and appreciable in terms of process yield and quality of the final oil extracted, based on cannabinoids such as CBD, CBG, THC. Furthermore, the lipophilic nature of the molecules that are intended to be brought into the olive and/or hemp oil make this process particularly efficient.

The yield of the process has been calculated between 75% and 85%, depending on the type of molecule most present in the solid matrix, the pressures exerted, the length of the pressure and depression cycles, the temperatures of the compound introduced in the extractor, and the operating temperatures of the entire extraction cycle.

Once the humid mass coming from the kneading has been introduced into the extractor, the extraction process begins, consisting of continuous cycles of pressure exerted by the piston on the humid mass and depression exerted by the removal of the piston from the humid mass which regains volume when the piston is retracted, bringing it back in its starting position. After waiting for the humid mass to regain its maximum volume and reabsorb part of the solutions that are flowing out of the holes in the extraction cylinder, a cycle of pressure and depression starts again. This process continues for a series of continuous pressure and vacuum cycles, until the maximum amount of polar and non-polar solvent used in the kneading process has been extracted.

Given the maximum amount of wet mass that can be inserted into the extractor, the amount of polar and non-polar solvent used in kneading, it is possible to program a defined set of pressure and depression cycles, and therefore compose a well-defined extraction program. It is therefore of interest to note that, on an industrial scale, the extraction method according to the present invention can be fully automated and managed by a suitable dedicated software. The extraction time with continuous cycles of pressure and depression of the moist mass coming from the kneading is variable, typically but not limited to from 1.5 to 2 hours.

The mixture which is extracted and collected in about 2 hours separates into three well-defined solution/suspension fractions: a first heavier fraction in the form of water colored by flavonoids, chlorophyll, sugars, vegetable proteins, initially present in the solid matrix, in the center a mixture of vegetable waxes, resins, terpenes, vegetable parts soaked in polar solvent (water) contained in the hemp flower, above that is the lighter fraction, i.e. the one represented by olive oil containing the main terpenes and cannabinoids present in the starting solid matrix, for example CBD and decarboxylated CBDA (in water during kneading), if the solid matrix contained CBD+CBDA and if the process is performed at temperatures and times such as to decarboxylate the CBDA.

The extracted fractions are then collected in a decanter. The decantation can take place, on a laboratory scale, with a common separator funnel or with similar structures that exploit the same technicalities if the step is carried out on an industrial scale.

The decantation can be performed at room temperature (18-20° C.) or alternatively at a controlled temperature.

In the decanter, a set of extraction fractions is collected including water, oil, cannabinoids, vegetable waxes, terpenes, flavonoids, sugars, carbohydrates, proteins which comes out of the extractor at a temperature between 60/90° C. As the heat exchange takes place between the decanter and the external environment, the following also takes place:
The clear separation of the two mutually immiscible solvents used in the process, water and immiscible apolar solvent.
The slow winterization of the vegetable waxes, which starts at around 45/50° C., so that the waxes, solidifying earlier, carry a large part of the small woody and vegetable residues present in the solution and partly impregnated with water, therefore heavier of their initial starting state (dry).
Separation between water-based solution (process by-product B), apolar solvent solution containing vegetable waxes, resins and fats, and microscopic vegetable particles (process by-product C), apolar solvent solution containing cannabinoids CBD, CBG, THC, terpenes.

At the end of the decantation, after about two hours, it is possible to separate the three fractions, for example by opening the tap located at the base of the decanter.

The following will then be retrieved in order of exit from the bottom tap:
Process by-product B: water-based polar solvent solution containing most of the water-soluble compounds present in the solid matrix, such as sugars, flavonoids, terpenes and/or terpenoids (terpenes modified by oxidation), enzymes, part of the chlorophyll, residues of the solid matrix.
Process by-product C: starting apolar solvent solution with a high concentration of waxes, resins, fats, and fat-soluble cannabinoids lighter than water, but heavier than the apolar solvent solution mixed with cannabinoids CBD, CBG, THC This fraction, in the form of suspension, is the one that is in direct contact with water.
Process product D: this solution still has a temperature above 50° C., so it is much less viscous than the room temperature and furthermore it is no longer just the apolar solvent used at the beginning of the process, but the same with dissolved therein 75%-85% of the cannabinoids present in the starting solid matrix.

In addition to the aforementioned product D and by-products B and C, there is a further by-product of the whole process, namely by-product A.

In more detail, at the end of the process we also obtain:
Process by-product A: solid matrix of Cannabis biomass, mixed with water residues and the apolar solvent used. This is the solid matrix waste definitively pressed at the end of the extraction process, which obviously still contains residues of water and olive oil, typically to the extent of about 5-10% of the initial process volumes.

The last step of the process according to the present invention is, as mentioned, the filtration step. Filtration takes place according to filtration standards known in the art.

During the experiments inherent in the definition of the process according to the present invention, olive oil (simple or extra virgin), hemp seed oil and a mixture of olive oil and hemp seed oil were used as apolar solvent.

It is also of interest to note that all the by-products obtainable with the process according to the present invention have features which make them particularly usable for specific applications of interest to the community.

It is also of interest to note that further by-products can be obtained from said process by-products B and process by-product C, and in particular a first secondary by-product B′ and a second secondary by-product C′.

In more detail, the first Process Secondary By-Product B is a semi-plastic biodegradable molasses-type paste of sugars, carbohydrates, vegetable proteins, enzymes, cannaflavins and other flavonoids, part of the terpenes present in the starting biomass, and the secondary process by-product C is a concentrated solution containing terpenes, starting apolar solvent, waxes, resins, fats, and a small part of the terpenes present in the starting biomass.

First of all, it is of interest to point out that the process according to the present invention offers various advantages. In more detail, it should be noted that:
advantageously, the decarboxylation of the fat-soluble molecules takes place in the kneading step in an almost complete way even if with longer times than the traditional methods, commonly used, but at lower temperatures than 110-120° C., values typically used in known decarboxylation ovens. In this way, the properties of many more thermolabile molecules present in Cannabis are kept unaltered, such as terpenes and flavonoids (23 are flavine cannas, i.e. the unique and typical flavonoids of Cannabis, identified up to now: they are water-soluble molecules and accumulate inside the cell vacuoles).

Advantageously, said process is such as to obtain yields similar to those of known processes but at considerably lower costs than other extraction processes, with the same plant size, even if the decarboxylation times of the cannabinoids CBDA, CBGA, THCA are longer.

Advantageously, the process in question is natural and ecological, not using chemical substances such as gas, alcohol or in any case synthetic substances.

Advantageously, the separation of the fractions obtainable following extraction, as well as following decanting, is clear and takes place in a relatively short time, allowing above all a total recovery of said fractions which are free from traces of the other fractions.

Advantageously, all the by-products obtainable with said process, in addition to the product of interest, i.e. olive and/or hemp seed oil with a high concentration of phytocannabinoids, can be used and further processed for various applications.

Advantageously, given the uniformity of the process, it should also be noted that: if the starting solid matrix has a high concentration of CBDA, or CBGA and a low % of total THC, typically less than or equal to 0.2%, also the by-products of process, A, B, C, and especially product D, will have a THC content of less than or equal to 0.2%. This aspect is very important as it makes it possible to never accumulate the psychoactive cannabinoid THC in any by-product of the process beyond the threshold of 0.2%.

Advantageously, the process plant is not as dangerous as plants that use supercritical gases, or ethyl alcohol, to extract cannabinoids from indica or sativa Cannabis biomass. The water does not catch fire, so there are fewer parameters to respect compared to other extraction processes and relating to safety standards such as anti-detonation and anti-flammability. The product process D, i.e. (extra virgin) olive and/or hemp seed oil represents about 70% of the starting apolar solvent, and contains high concentrations of cannabinoids such as CBD, CBG, THC (respectively if the starting solid is with a high concentration of CBDA, CBGA, or THCA) in the extent of about 75-85%, of the cannabinoids initially present in the solid matrix. The 15/25% of the cannabinoids present in the starting solid matrix remain in minimal part in the solid matrix, in the aqueous solution (in an even minimal part), and in the process by-product C.

In the case of olive oil as an apolar solvent, a solution based on olive oil with a high concentration of cannabinoids will typically be obtained as Process product D, precisely to the extent of approximately 80% of the cannabinoids present in the starting solid matrix. Suitably filtered, it is possible to bring this concentration to a higher level, about 85% in cannabinoids, since further vegetable residues, chlorophyll, waxes and resins present in minimal part in the Process D product are eliminated. The filtered Process D product can be offered on the market as CBD oil, or CBG, or other fat-soluble cannabinoids (even at high concentrations of THC in compliance with the regulations of the reference country), depending on the type of starting solid matrix, for food use, or for industrial cosmeceutical use.

It is also of interest to point out that: in addition to the fat-soluble cannabinoid initially present in the starting solid matrix, a high % of terpenes end up in Process product D, other molecules present in the starting solid matrix. This liposoluble solution has a low concentration of water-soluble substances present in the starting solid matrix, such as for example chlorophyll, especially if hemp seed oil is used as apolar solvent: the water-soluble substances have been dissolved in the polar solvent used in the process, especially water due to its low cost. This final CBD oil, or CBG, or in combination with THC, with its high concentration of terpenes, determines an appreciable entourage effect: a synergistic result that can significantly modify the action of the main active ingredients, improving their action and minimizing minimize possible side effects. It is in fact known that some terpenes, for example, bind with neurotransmitters such as the CB1 and CB2 receptors, influencing various functions of the human body as well as its response to external agents. Others seem to modify the permeability of cells by modulating, for example, the assimilation of THC. Still others interact with the release of dopamine and serotonin. Therefore the Process product D represents the Primary Product of the entire process, i.e. a CBD oil (for example if one starts from a solid matrix with a high % content of CBD+CBDA and a low % content of THC+THCA<0, 5%) which in addition to containing the decarboxylated cannabinoid CBDA, contains high % of terpenes, low % of flavonoids, low % of chlorophyll, and low % of waxes and resins which are captured by Process By-Product C. Furthermore, if during the kneading process some washings of the biomass are carried out with water, before introducing the apolar solvent, it is possible to eliminate many sugars, carbohydrates, flavonoids, enzymes, proteins, which will be separated from the fat-soluble cannabinoids. Given the yields of the process, given the concentrations of cannabinoids present in the starting solid matrix, by varying the quantity of solid matrix and the quantity of apolar solvent used, it is possible to estimate the final concentration of cannabinoids which will be present at the end of the process in the various Process by-products A, B, C, and product D. Consequently it is possible to obtain a final product, process product D, i.e. olive and/or hemp seed oil, with the desired quantities of cannabinoids in the apolar solvent (for example CBD oil at 5%, 10%, 15%, 20%) and at the same time to also manage the concentrations of THC in the various process by-products, a very important aspect in order not to accumulate psychotropic molecules such as THC beyond certain percentage thresholds established by the various local territorial laws.

It is in any case of interest to point out that the same process according to the present invention can be used, in countries where the laws allow it, to obtain a Process D product with a high concentration of THC or other psychoactive and fat-soluble cannabinoid, or from galenic pharmacies for the preparation of THC-based oils for medical cannabis-based therapies.

In summary, the extractive process according to the present invention leads to obtaining a product in which it is possible to have an appreciable concentration from a therapeutic point of view of the main desired cannabinoids, by initially selecting the type of solid matrix from which to extract. Said oil also contains a large quantity of terpenes (from 10 to 90 times greater than currently marketed cannabinoid-based oils. It is free from vegetable waxes; it is free from synthetic chemical solvent residues; it is different in flavor from the cannabinoid-based oils currently on the market, obtained with the various known processes; it contains a part of flavins (water soluble) which bind to the flavonoids present in olive oil or hemp seed oil, it can be deprived of sugars, proteins, enzymes whose degradation can shorten the shelf life of the product D.

Finally, it is of interest to point out that the starting solid matrix, i.e. the biomass of Cannabis, must have high quality standards, in terms of the absence of mould, insecticides, pesticides or heavy metals.

### DETAILED DESCRIPTION OF THE INVENTION

The process for obtaining an oil based on phytocannabinoids according to the present invention provides, in all its embodiments, that:
the Solid Matrix is represented by biomass of intact or shredded flowers and leaves in particle size between 0.5 mm, typically 1 mm and 10 mm. The biomass may also be introduced as intact flowers and leaves since during kneading, such biomass is broken up and reduced into smaller parts. It is advisable to avoid the presence of large branches. The solid matrix must not have a powdery character, as a powdery solid matrix would make the process less efficient in terms of quantity, quality and in terms of quantity of cannabinoid molecules transported from the solid matrix to the apolar solvent. Also, generally, depending on the process standards, the residual moisture of the Cannabis biomass is <12%.

The type of biomass of Cannabis or hemp to be used depends on the concentration of the major cannabinoid that is desired to have within the apolar solvent chosen at the end of the process, in particular in the process product D. If one wants a CBD oil, a biomass with a high content of CBD+CBDA should be chosen, if one wants a CBG oil, a solid matrix with a high content of CBG+CBGA will be used, or THC+THCA if one wants an apolar solvent with a high concentration of THC, or other fat-soluble cannabinoid molecules that one wants to transport in the apolar solvent used. By choosing the appropriate solid starting matrix, a certain amount of concentrated oil will be obtained with CBD, CBG, THC, or any other desired fat-soluble cannabinoid. So the Solid Matrix can be: Cannabis with a higher concentration of CBD+CBDA, or CBG+CBGA, or THC+THCA, THCV+THCVA, and so on, in short, a biomass of Cannabis, sativa or any indica.

The polar solvent is water, given its cheapness and its physical-chemical behavior in contact with oils and waxes, and in particular with the solid Cannabis matrix. The water can be tap, demineralized, ionized, osmotic.

The apolar solvent is preferably extra virgin olive oil and/or hemp seed oil, or other apolar solvent immiscible with water.

The process according to the present invention provides for the following steps to be carried out in sequence: kneading; extraction; settling; filtration.

All the elements of the machines in contact with the solid matrix and/or with the solvents are in food grade stainless steel and the finishing standard of the machines is in pharmacological GMP.

In one of its preferred embodiments, the oil obtainable with the process according to the present invention is prepared using as ingredients: 1 Kg of Cannabis flowers with grain size between 0.5 and 10 mm, and Total CBD=10%, THC<0.2%; from 2 to 5 liters of tap water; 1 Lt of non-polar solvent, in this case: Olive oil.

Machines and tools used are by way of non-limiting example the following. In any case, it is of interest to underline that it is taken for granted that the person skilled in the art considers different instrumentation equally usable but functionally in an equivalent or similar manner for carrying out said process steps.

### Example of Type of Instrumentation Used

Horizontal mix kneader-cooker.
Hydraulic press from 0 to 50 tons.
Liquid collection surface in stainless steel placed on the support surface of the press.
Stainless steel basket with micro-perforated walls.
Cylindrical base of the basket, also micro-perforated.
Upper cap in stainless steel with the same internal diameter as the basket and with the upper part that can be fitted into the piston of the hydraulic press.
Bag of micro-perforated fiber mesh with holes between 100 and 200 microns. This bag has the same internal dimensions as the extractor cylinder.
Funnel separator tank, in stainless steel.
Specific laboratory HPLC for cannabinoid analysis, capable of performing the analysis in 10-30 minutes.
Water distiller.

In an example of its preferred embodiments, the process according to the present invention provides for:
introducing Cannabis flowers into the kneading machine and starting the mixing blades, setting their rotation speed to a minimum, preferably at a speed of 10-20 rpm and leaving to knead for 2-5 minutes;
adding water, letting the kneading continue at a speed of 10-20 rpm for a time interval of between 5-10 minutes;
increasing the mixing speed of the blades to 20-35 rpm, and temperature setting between 60° C.-75° C. When the internal temperature of the mass being kneaded has reached 60° C.-75° C., approximately 3 liters of water are drained from the kneader, and another 3 liters of clean water are added at a temperature of 60° C.-75° C. Continue kneading for another 15-20 minutes and repeat the same operation of replacing the water. The drained water is filtered and conveyed into the distiller tank, the same in which the process by-product B will then be collected, suitably filtered;
increasing the mixing speed of the blades to 20-35 rpm, and setting the temperature between 90° C.-110° C. When the internal temperature of the mass being kneaded has reached 90° C.-110° C., kneading continues at these temperatures for 2-4 hours. After 1.5-2 hours, a sample of the compound is taken from the kneader and analyzed by HPLC to verify the degree of decarboxylation of the CBDA. If the CBDA molecules are not yet completely decarboxylated, the kneading is left to continue for a further 1-2 hours, or for the time necessary to reach the desired decarboxylation;
the lowering of the internal temperature of the kneader to 60° C.-78° C. and reduction of the kneader speed to 20 rpm, and introduction of the apolar solvent, for example extra virgin olive oil;
continuation of kneading for 40-60 minutes;
shutdown of the kneader and transfer of the compound into the extractor cylinder; transferred all the mixture from the kneader to the cylinder, and closing the extractor;
initiation of the compression and depression cycles of the mass to be extracted by applying pressure exerted by a plate provided with a piston inside the extractor which moves vertically up and down, compressing and depressing the mass of decarboxylated Cannabis leaves and flowers and recovering from time to time the extract into a vessel for the separation of the extraction fractions. The compression and vacuum cycles are to be carried out until the compressed mass loses its elasticity which is obtained after eight pressure-compression cycles, preferably after 16 pressure-compression cycles and even more preferably after 20 pressure-compression cycles. Typically 8-10 pressure-compression cycles are performed in a time interval that varies from 10 to 15 seconds. The pressure is exerted by a press-like plate weighing 2-3 tons. In more detail, the compression and depression cycles are performed until no more liquid is observed coming out of the extractor;
after having completely transferred the extracted liquid into the separator, the extract is left to decant for a time interval of at least 1 hour and preferably for two hours;
subsequently the three extracted fractions are recovered, by-products B and C of the process and the product D of the process corresponding to the desired product, i.e. the phytocannabinoid-based olive oil.

Said oil is further filtered and percolated with a multiple pleat food paper filter, filtration degree 45-100 micron, by percolation, a process which is carried out at 16-18° C., even for 12 hours, for 1 L of CBD oil.

From the filtration by percolation of Process Sub-Product D, approximately 600-650 ml of CBD oil are obtained, dark amber, glossy, and with a CBD content of 7.5-8.5%.

Like all cannabinoid-based products, the product obtainable with the process according to the present invention is suitable for use in the therapeutic treatment against neurodegenerative diseases, anxiety, nausea, depression, rheumatoid arthritis, acne, psoriasis, multiple sclerosis.

1. Extractive process for obtaining an oil based on phytocannabinoids, said oil being selected from olive oil, extra virgin olive oil, hemp seed oil, seed oil in general and combinations thereof, said process using dried flowers and leaves of the Cannabis plant as starting biomass, said process providing for the sequential implementation of the following steps of: kneading, extraction, decantation/separation, filtration; said process being characterized in that it uses as an extraction solvent water as polar solvent jointly with an oil selected from among said olive oil, extra virgin olive oil and hemp seed oil, seed oil in general and combinations thereof as apolar solvent, said starting biomass including substances such as CBD, CBDA, CBG, CBGA, THC, THCA, THCV, THCVA, said kneading step providing for inserting said dry biomass matrix of Cannabis flowers and leaves into the kneader provided with a stirring system, the latter being variously divided, and subsequently inserting said polar solvent and increasing the temperature from 0° C. up to 95° C., said insertion of the polar solvent occurring at a temperature in said range 0° C.-95° C., said polar solvent being replaceable several times when the temperature of the compound in the kneader is in the range of 60° C.-75° C., said increase taking place progressively bringing the temperature up to no more than 95° C., for a time interval of between 1 and 4 hours, said heating, when it occurs in the range from 70° C. to 95° C., promoting the decarboxylation of the starting cannabinoids present in said biomass; said process subsequently providing for the reduction of the kneading temperature up to 65° C. and the introduction into the kneader of said apolar solvent to then continue kneading for a time interval of between 40 minutes and 60 minutes, said process subsequently providing for the transfer of the product of the kneading, the latter being a dry matrix decarboxylated with said polar and apolar solvents, inside an extractor, said process providing that the extraction step includes the execution of compression and depression cycles of the wet mass coming from the kneading step, said compression occurring by virtue of the pressure action of a piston system with which said extractor is provided, said depression occurring from the lifting/retraction of said piston system from said wet mass, said process providing that the extraction step takes place in a time interval of between 1.5 hours and 2 hours; said process providing that the extract is transferred inside a decanter/separator container, said extract comprising: a first heavier fraction in the form of colored water comprising sugars, vegetable proteins, carbohydrates, cannaflavins, flavonoids and chlorophyll and initially present in the solid matrix, above a mixture of vegetable waxes, resins, terpenes, vegetable parts soaked in polar and apolar solvent contained in the hemp flower, above the lighter fraction, the latter being said apolar oil, containing terpenes and cannabinoids present in the starting solid matrix, said process then providing that the decantation takes place at room temperature, the latter being between 18-25° C., said decantation promoting the formation of a process by-product B, a process by-product C and a process product D, said process by-product B being a water-based polar solvent solution comprising sugars, flavonoids, terpenoids, enzymes, part of the chlorophyll, residues of the solid matrix, the latter being a process by-product A; said process by-product C being a suspension based on waxes, resins, fats and fat-soluble cannabinoids, said process product D being an oil selected from extra virgin olive oil, olive oil, hemp seed oil, oil of seeds in general and combination thereof with a quantity of between 75%-85% by weight of the cannabinoids present in the starting solid matrix dissolved therein, said process providing for the recovery of said process product D and its subsequent filtration.

2. Process according to claim 1, wherein the starting solid matrix comprises dried flowers and leaves of the Cannabis plant with a particle size of between 0.5 mm and 10 mm.

3. Process according to claim 2, wherein, in the kneading step, the polar solvent water is introduced at a room temperature of about 25° C.

4. Process according claim 1, wherein the polar solvent water is drinking water selected from tap water, demineralized water, osmotic water, and the polar solvent is olive oil.

5. Process according to claim 1, wherein, during the kneading step, before the addition of the apolar solvent, and when the temperature of the compound in the kneader has reached a temperature of 50° C.-75° C., part of the polar solvent is drained and replaced with new polar solvent.

6. Process according to claim 1, wherein, during the kneading step, the starting solid matrix is made to knead with water for a time ranging from 1 hour to 4 hours between 70° C. and 95° C., not beyond the room temperature of water, and subsequently the temperature is brought between 60° C. and 85° C., to then add said apolar solvent and continue kneading for a further 30-70 minutes.

7. Process according to claim 1, wherein the biomass matrix of dried Cannabis flowers and leaves comprises a quantity of THC lower than or equal to 0.2% by weight.

8. Process according to claim 1, wherein the starting biomass has a quantity of moisture lower than 12% by weight, said biomass being represented by 1 Kg of Cannabis flowers with a particle size of between 0.5 and 10 mm, said biomass having a total quantity of CBD of 10% by weight, THC <0.2% by weight; said process using from 2 to 5 L of tap water; and 1 L of apolar solvent in olive oil.

9. Process according to claim 1, wherein the biomass matrix of dried Cannabis flowers and leaves comprises a greater amount of THC+THC, or CBG+CBG, or CBD+CBDA, THCV+THCVA, or other fat-soluble cannabinoid.

10. Product comprising an oil selected from olive oil, extra virgin olive oil, hemp seed oil, seed oil in general and combinations thereof, including CBD, CBG, THC, terpenes, flavins, wherein the CBD content ranges from 7.5% to 8.5% for 600ml-650ml of oil.

11. Product according to claim 10, wherein the oil is olive oil.

12. Product according to claim 10, wherein the oil is extra virgin olive oil.

13. Product according to claim 8 for use in a method of therapeutic treatment of neurodegenerative diseases, anxiety, nausea, depression, rheumatoid arthritis, acne, psoriasis, multiple sclerosis.
